(19) United States
(12) Patent Application Publication
(10) Pub. No.: US 2017/0113005 A1
(43) Pub. Date: Apr. 27, 2017

(54) DEVICE FOR TRANSPLANTING BODY FAT

(71) Applicant: Human Med AG, Schwerin (DE)

(72) Inventors: Bernd LINDNER, Stockelsdorf (DE); Christian WRETH, Leezen (DE); Felix BABINSKY, Schwerin (DE); Frank NIKLAS, Lubz (DE); Konrad-Wenzel WINKLER, Warin (DE); Andreas RUNOW, Schwerin (DE); Gernot SCHLEE, Schwerin (DE)

(73) Assignee: Human Med AG, Schwerin (DE)

(21) Appl. No.: 15/298,889

(22) Filed: Oct. 20, 2016

(30) Foreign Application Priority Data
Oct. 22, 2015 (DE) 10 2015 118 011.1

(51) Int. Cl. A61M 5/315; A61M 5/19; A61M 5/36; A61M 5/31

(52) U.S. Cl. CPC A61M 5/31511; A61M 5/3145; A61M 5/19; A61M 5/3158; A61M 5/36; A61M 2005/3123

(57) ABSTRACT

A device for transplanting body fat includes a chamber in the form of a hollow cylinder divided by a plunger guidable on an inner casing surface of the chamber into a proximal portion and a distal portion which is fluidically decoupled from said proximal portion. A piston connected to the plunger has an end which has a fluidic connection extending through the plunger to the chamber, and the plunger is configured as a filter in the region of the fluidic connection, and the distal end of the piston comprises a first closable media connection and means for guiding fluids and/or fatty tissue are connectable to the chamber, wherein fluid and/or fatty tissue can be sucked into the proximal portion of the chamber and/or in through the plunger by supplying the first media connection in the open state with a negative pressure.

### BACKGROUND OF THE INVENTION

The invention relates to a device for transplanting body fat and a system having the device.

The person skilled in the art knows that body fat can be aspirated, disposed of, or further used. With respect to further use, transplanting of body fat is frequently considered. The devices and methods required for this are generally known to the competent person skilled in the art.

Frequently, a water jet is first introduced into the body at an appropriate pressure, in order to separate fatty and connective tissue. For example, a tumescent solution is injected. As a result, fat cells can be dislodged from existing connective tissue. The freed fatty tissue is then aspirated, processed (for example desiccated) and injected or re-injected into a patient.

However, with respect to the known devices and methods the disadvantage is that different equipment is required for the individual method steps.

It is therefore the object of the present invention to indicate a device for transplanting body fat, which is simple, safe and flexible, and with which the transplanting of body fat is completely feasible.

This object is achieved by the subject matter of the independent claims 1 and 9.

### SUMMARY OF THE INVENTION

The subject matter of the present invention is a device for transplanting body fat, comprising a chamber which, at least in sections, is in the form of a hollow cylinder, said chamber being divided by a plunger which can be guided on an inner casing surface of the chamber into a proximal portion and a distal portion which is fluidically decoupled from said proximal portion, wherein a piston is connected to the plunger, said piston comprising a distal end which has a fluidic connection extending through the plunger to the proximal portion of the chamber, and the plunger is configured, at least in sections, as a filter in the region of the fluidic connection, and the distal end of the piston comprises a first closable media connection and, furthermore, means for guiding fluids and/or fatty tissue are or can be connected to the proximal portion of the chamber, wherein fluid and/or fatty tissue can be sucked into the proximal portion of the chamber and/or fluid can be sucked in through the plunger by supplying the first media connection in the open state with a negative pressure. According to the invention, it is envisaged that the distal portion of the chamber comprises a second media connection, so that fluid and/or fatty tissue can be pumped out by means of a displacement of the plunger through the proximal portion of the chamber by supplying the second media connection with a positive pressure while the first media connection is closed, and/or fluid and/or fatty tissue can be pumped out by means of the displacement of the plunger through the proximal portion of the chamber and, simultaneously, fluid can be discharged through the plunger by supplying the second media connection with a positive pressure while the first media connection is at least open.

The advantage of this is that the device according to the invention is particularly simple and safe and has such a flexible design that all of the steps of the method for transplanting body fat are feasible therewith.

In order to illustrate these advantages, a method for transplanting body fat according to another aspect of the present invention is described below.

The meanings of the terms ‘proximal’ and ‘distal’ are generally known to the person skilled in the art. Nevertheless, an explicit definition is provided here for reasons of clarity. The term ‘proximal’ is defined in connection with the present invention as being located towards the center of the body of a patient. The term ‘distal’ is defined in connection with the present invention as being located away from the center of the body of the patient. In the case of device features, account is therefore to be taken of whether a particular feature is provided for use closer to the center of the body of the patient or further away therefrom compared with another feature.

In a first method step, an infiltration fluid is initially provided such that it can be introduced under pressure into the region of a location for harvesting body fat. The device according to the invention for transplanting body fat can be used in a particularly advantageous manner for this purpose. Thus, infiltration fluid can be sucked into the proximal portion of the chamber which is in the form of a hollow cylinder particularly easily and safely. For this purpose, the device for transplanting body fat can, for example, be guided with the proximal portion into a reservoir of infiltration fluid and can then be supplied with a negative pressure via the open first media connection. This therefore ensures that the device can be easily and safely used. The device can also be handled particularly well, thus increasing safety in a medical environment. The device can also be held in the reservoir of infiltration fluid and can suck in the latter, without having to connect means for guiding fluids and/or fatty tissue to the proximal portion. A large flow cross-section can therefore be advantageously realized and rapid filling is made possible. A position of the piston of the device in a distal dead-center position or at another position within the chamber which is in the form of a hollow cylinder can be advantageously adjusted. Thus, the amount of infiltration fluid to be sucked in can be advantageously easily metered. Another advantage is that excess infiltration fluid can be easily discharged through the filter of the plunger and the first media connection. A further advantage is that the first media connection can be opened and closed. The device can therefore be switched over particularly reliably between states where it is possible and not possible to supply pressure externally via the first media connection.

In a second method step, the infiltration fluid is introduced into the region of the harvesting site of the body fat with a positive pressure of preferably 0.5 bar to 30 bar, more preferably 1 bar to 5 bar and, particularly preferably, 1 bar to 2 bar, in order to mobilize said body fat. The fatty tissue is loosened by the infiltration fluid, making it easier to dislodge it from the connective tissue. This latter process can be carried out, for example, by aspiration. The device according to the invention can be advantageously adapted to different infiltration tasks with different means for guiding fluids and/or fatty tissue, which can be connected to the proximal portion of the chamber, and is as a result particularly flexible. For example, the means can comprise an infiltration cannula having, for example, a 300 μm nozzle diameter so that the infiltration fluid can be introduced in a particularly simple and safe manner. The first media connection of the device can be advantageously closed in a simple manner and the second media connection can be supplied with a positive pressure, in order to act on the infiltration fluid with a defined positive pressure. Since the proximal portion and the distal portion are fluidically decoupled, the plunger is displaced in a particularly reliable manner, without the pressure medium being able to enter the proximal portion. During the infiltration, the device can also be handled particularly easily and safely, increasing safety.

In a third method step, the dislodged fatty tissue is aspirated. The aspiration is preferably effected at a negative pressure of preferably −900 mbar to −300 mbar, particularly preferably of −600 mbar to −400 mbar and, very particularly preferably, of −500 mbar. The device can advantageously be flexibly equipped with suitable means for guiding fluids and/or fatty tissue. For example, an appropriate aspiration cannula having, for example, a 2 mm nozzle diameter can be mounted at the proximal portion of the chamber. The process of aspiration can be realized particularly easily and safely with the device according to the invention. To this end, the first media connection can be advantageously easily opened and can be acted upon by a negative pressure. Furthermore, the advantage of the device according to the invention is that, during the process of aspirating fatty tissue and fluid by means of the plunger, which is configured, in sections, as a filter, fluid can be sucked in through the plunger, wherein fatty tissue remains in the proximal portion, so that only the fluid can be discharged through the first media connection. Thus, the fatty tissue or a mixture of materials, comprising at least the fatty tissue and the fluid, can be aspirated and processed in a single method step with the device according to the invention.

The fourth method step consists of reinjecting the body fat. The reinjection is effected at a positive pressure of preferably 0.5 bar to 30 bar, more preferably 10 bar to 15 bar, particularly preferably 6 bar to 9 bar and, very particularly preferably, 7 bar. The advantage of the device according to the invention is that a reinjection can be effected particularly easily and safely. Body fat can thus be easily and safely yielded from the proximal portion of the chamber at a defined pressure, by supplying the second media connection with a positive pressure. The device can be adapted particularly easily and flexibly to different reinjection tasks. The means for guiding fluids and/or fatty tissue can therefore comprise, for example, a reinjection cannula having a nozzle diameter of, for example, 1.4 mm. Particularly advantageously, it is possible to use the device according to the invention to pump the body fat out of the proximal portion of the chamber, simultaneously separating the fluid and fatty tissue. To do this, the first media connection merely has to be opened. As pressure builds up in the proximal chamber as a result of a displacement of the plunger, the mixture of materials in the proximal portion, which can contain a residual content of fluid and the fatty tissue, is pressed against the plunger which is configured, in sections, as a filter. The remaining fluid can therefore be separated from the fatty tissue and discharged through the filter particularly easily and safely, whilst only the fatty tissue can be pumped out of the proximal portion into, for example, a region of an implant site. Therefore, the processes of reinjection and processing can also be advantageously combined with the device according to the invention. It is also advantageously possible to additionally act on the first media connection with a negative pressure. Thus, a pressure difference in the fluidic connection between the proximal portion of the chamber and the distal end of the piston, i.e. a pressure difference across the plunger and, therefore, the filter effect thereof, is to be increased. The desiccation effect for the fatty tissue can be advantageously further improved in this way. This also advantageously helps to ensure that body fat can be transplanted particularly quickly and, at the same time, safely and easily with the device according to the invention.

Preferred embodiments of the device according to the invention are described below. In order to illustrate the advantages of the device, reference is occasionally made to the method according to the invention described above. If necessary, preferred embodiments of the method according to the invention are also described.

It is now envisaged in a preferred embodiment of the device according to the invention that the plunger has at least one additional fluidic connection between an outer casing surface of the plunger and an outlet opening at the distal end of the piston.

The advantage of this is that a positive pressure, which arrives as a result of manufacturing tolerances or other fault effects from the distal portion of the chamber between the inner casing surface of the chamber and the plunger, can be discharged by this additional fluidic connection into the environment. The safety of the device is advantageously increased as a result, since mixing of the pressure medium with fatty tissue in the proximal portion is always avoided.

In another preferred embodiment of the present invention, it is envisaged that at least one antechamber having at least one bypass opening and/or at least one cannula or cannula having a bypass opening is/are or can be connected to the proximal portion of the chamber as the means for guiding fluids and/or fatty tissue.

The advantage of this is that aspiration of fatty tissue from the region of the harvesting site via the bypass opening can be interrupted and continued in a flexible manner. This advantageously increases the flexibility and also the safety of the device. The possibility of mounting different cannulas on the device means that the latter can be adapted in a flexible manner to an extremely wide range of requirements from the aforementioned method steps of fluid absorption, infiltration, aspiration of fatty tissue and injection of fatty tissue. The cannulas to be provided for this purpose are generally known to the person skilled in the art.

In another preferred embodiment of the present invention, it is envisaged that the antechamber comprises at least one prefilter for filtering out connective tissue residues.

The advantage of this is that an agglomeration of connective tissue residues and, associated therewith, the risk of clogging of the device are avoided. The reliability and safety of the device are thus advantageously increased.

In another preferred embodiment of the present invention, it is envisaged that the second media connection comprises a valve which is in a flow-blocking state when the piston is in the distal dead-center position, and is in a flow-enabling state when the piston is not in a distal dead-center position.

The advantage of this is that due to the fact that the valve is in a flow-blocking state a vacuum which is generated by means of the first media connection in the proximal portion of the chamber is not disrupted, even if undesirable leaks occur in the region of the plunger and of the inner casing surface of the chamber. In other words, if an undesirable fluidic connection between the proximal portion of the chamber and the distal portion of the chamber is created between the plunger and the inner casing surface of the chamber as a result of fault effects, for example, the vacuum can still not be destroyed by a medium streaming in through the second media connection because the valve is in the flow-blocking state. Furthermore, it is advantageously ensured by the fact that the valve is in the flow-enabling state that positive pressure introduced into the distal portion of the chamber can be safely discharged again by means of the second media connection.

In another preferred embodiment of the present invention, it is envisaged that the piston can be latched at a distal dead-center position by being rotated about its longitudinal axis.

The advantage of this is that it is possible to securely lock the piston. This is particularly advantageous, for example, if the first media connection is acted on with a negative pressure or fluid and/or fatty tissue located in the proximal portion of the chamber is/are to be retained in the chamber.

In a particularly preferred embodiment, the valve of the second media connection can be switched into its flow-blocking state by rotating the piston about its longitudinal axis at the distal dead-center position and, at the same time, the piston can be locked. This significantly enhances the safety of the device.

In another preferred embodiment of the present invention, it is envisaged that a transmission limit of at least one portion of the plunger which is configured as a filter is between 100 μm and 300 μm, particularly preferably between 100 μm and 250 μm and, very particularly preferably, between 100 μm and 190 μm and/or a transmission limit of at least one prefilter is between 0.5 mm and 3 mm, particularly preferably between 1 mm and 2 mm and, very particularly preferably, of 1.5 mm.

The advantage of this is that both reliable prefiltering of larger connective tissue residues and a reliable desiccation of the fatty tissue are possible. Studies have shown that these transmission limits simultaneously and advantageously allow a sufficiently large volume flow into and, where appropriate, through the device.

In another preferred embodiment of the present invention, the second media connection can be acted on with a positive pressure between 0.5 and 30 bar, preferably between 1 and 15 bar, more preferably 1 bar to 5 bar and, particularly preferably, 1 bar to 2 bar and/or the first media connection can be acted on with a negative pressure between −900 mbar and −300 mbar, preferably of −500 mbar.

The advantage of this is that the infiltration and aspiration method steps can be performed particularly well and safely with the device during the transplanting of body fat.

In another preferred embodiment of the present invention, the second media connection can be acted on with a positive pressure between 0.5 and 30 bar, preferably between 10 and 15 bar and, particularly preferably, of 7 bar.

The advantage of this is that the reinjection method step can be performed particularly well and safely with the device during the transplanting of body fat.

Another aspect of the present invention relates to a system, comprising at least one device according to the invention for transplanting body fat and at least one media supply device.

The advantage of this is that supplying the device with negative pressure, positive pressure or ambient pressure can be effected particularly easily and safely. Particularly advantageously, a supply with various media can be flexibly adjusted to the requirements. Purely by way of an example, a possible medium for supplying a positive pressure can be carbon dioxide or purified compressed air. Purely by way of an example, a vacuum pump can be used to supply a negative pressure. The media supply device can, for example, have valves to establish a connection with the environment in order to supply ambient pressure. Particularly advantageously, the system is also scalable since, for example, multiple devices can be provided for transplanting body fat.

In a preferred embodiment of the present invention, the system additionally comprises means for separating gaseous media flows and liquid material flows, which are arranged in an operative connection between the device for transplanting body fat and the media supply device.

The advantage of this is that the system itself is particularly safe and reliable if, for example, fluid is aspirated through the plunger of the device and the first media connection.

Other preferred embodiments of the invention are set out in the other features indicated in the subordinate claims.

The various embodiments of the invention indicated in this application can, unless otherwise indicated in individual cases, be advantageously combined with one another.

### BRIEF DESCRIPTION OF THE DRAWINGS

The invention is explained below in embodiment examples with reference to the associated drawings, wherein:

FIG. 1 shows a cross-sectional representation of a device according to the invention for transplanting body fat in a preferred embodiment,

FIG. 1A shows a side view of the device of FIG. 1,

FIG. 1B shows a top view of the device of figure, and

FIG. 2 shows a schematic representation of a system according to the invention in a preferred embodiment.

### DETAILED DESCRIPTION OF THE INVENTION

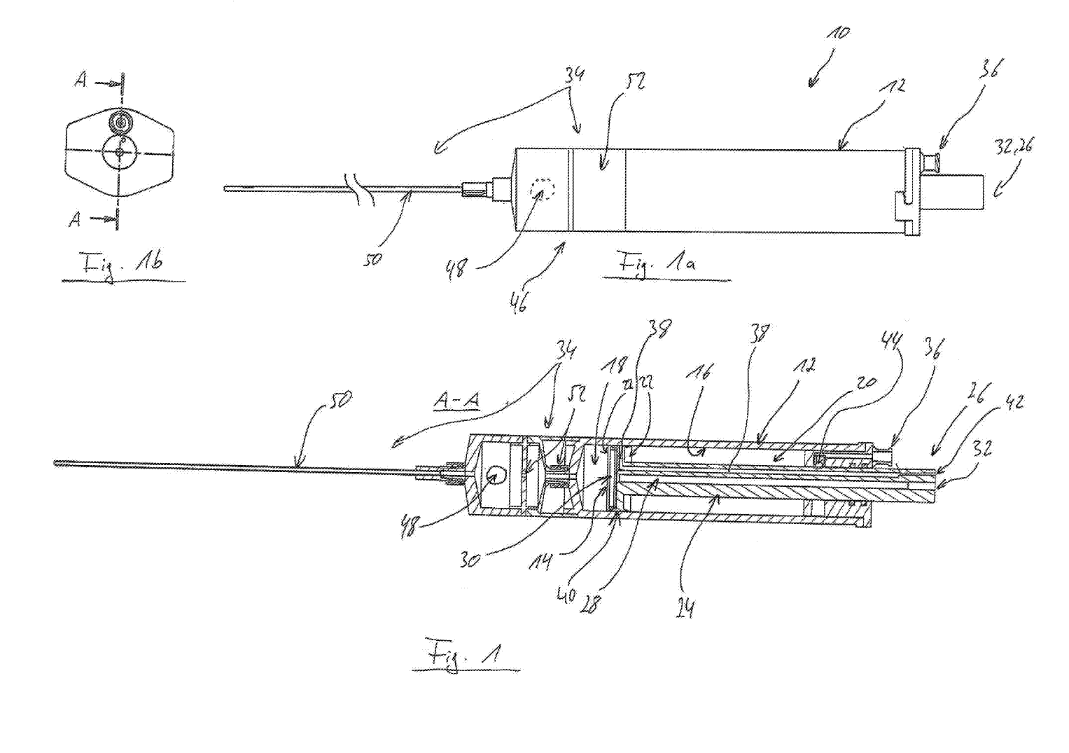

FIG. 1 shows a schematic representation of a device according to the invention for transplanting body fat in a preferred embodiment. FIG. 1A shows the device according to the invention for transplanting body fat 10 in a side view, FIG. 1B shows a top view. A chamber 12 which is substantially in the form of a hollow cylinder can be seen. As is clearly shown in the section view, the chamber 12 which is in the form of a hollow cylinder is divided by a plunger 14 which can be guided on an inner casing surface 16 of the chamber 12, which is in the form of a hollow cylinder, into a proximal portion 18 and a distal portion 20 which is fluidically decoupled from said proximal portion. The fluidic decoupling is brought about by the plunger 14 which comprises appropriate sealing lips 22. One sealing lip which faces the proximal portion 18 of the chamber 12 is configured in this embodiment example to prevent fluid from the proximal portion 18 penetrating a region between the plunger 14 and the inner casing surface 16. A sealing lip which faces the distal portion 20 of the chamber 12 is configured in this embodiment example to prevent compressed air or carbon dioxide from the distal portion 20 penetrating the region between the plunger 14 and the inner casing surface 16. A piston 24 is connected to the plunger 14. The piston 24 comprises a distal end 26 which has a fluidic connection 28 extending through the plunger 14 to the proximal portion 18. The plunger 14 is configured in sections as a filter 30 in a region of the fluidic connection 28, which faces the proximal chamber 18. The filter 30 has a transmission limit of 200 μm. The distal end 26 of the piston 24 comprises a first closable media connection 32, which is designed to be supplied with a negative pressure between −900 mbar and −300 mbar. A supply of ambient pressure is also possible. The closable media connection 32 can, for example, be a shut-off valve which is not shown here.

Furthermore, means 34 for guiding fluids and/or fatty tissue are exchangeably connected to the proximal portion 18 of the chamber 12. Fluid and/or fatty tissue can be sucked into the proximal portion 18 of the chamber 12 by supplying the first media connection 32 in an open state with a negative pressure. In addition, fluid can also be sucked in through the plunger 14, where the latter is configured as a filter 30. Furthermore, the distal portion 20 of the chamber 12 comprises a second media connection 36, which is designed to be supplied with a positive pressure between 0.5 and 30 bar. A supply of ambient pressure is also possible. By supplying the second media connection 36 with a positive pressure, while the first media connection 32 is closed, for example by means of the shut-off valve, a pressure can be built up in the distal portion 20. As a consequence of the pressure, the plunger 14 can be displaced, as a result of which a volume of the proximal portion 18 is reduced. Consequently, fluid and/or fatty tissue can be pumped out of the proximal portion 18 of the chamber 12. The plunger 14 has another fluidic connection 38 between an outer casing surface 40 of the plunger 14 and an outlet opening 42 at the distal end 20 of the piston 24. The additional fluidic connection 38 guarantees that a prevailing positive pressure in the distal portion 20 of the chamber 12 cannot escape into the proximal portion 18 of the chamber 12 even if, for example as a result of manufacturing tolerances or other fault effects, the plunger 14 cannot be guided by means of its outer casing surface 40 and the sealing lips 22 in such a way that it is completely pressure-tight against the inner casing surface 16 of the chamber 12. As a result of such fault effects, any medium which makes it way between the inner and outer casing surfaces 16, 40 is always discharged into the environment by means of this additional fluidic connection 38. If, at the same time as the second media connection 36 is supplied with positive pressure, the first media connection 32 is at least open, fluid can additionally be discharged through the plunger 14, while fatty tissue and, if necessary, a residual content of fluid can be substantially discharged in the opposite direction to the fluid, which can be discharged through the plunger 14, out of the proximal portion 18 of the chamber 12 in the direction of the means 34 or through these. Furthermore, the second media connection 36 comprises a valve 44 which is in a flow-blocking state, if the piston 24 is in a distal dead-center position and the piston 24 is latched there by being rotated about its longitudinal axis. The valve 44 can, purely by way of example, be executed as a ball valve, in which in a flow-blocking position the ball is pressed by a spring into a recess of the piston 24 and the valve 44 can thus be closed. By rotating the piston 24, the ball and the recess of the piston 24 are aligned with respect to one another or detached from one another by counter-rotation. The valve 44 blocks or allows medium to flow in through the second media connection 36 into the distal portion 20 of the chamber 12. If the piston 24 is not located in its distal dead-center position, the valve 44 is in a flow-enabling state. The means 34 for guiding fluids and/or fatty tissue comprise, in this embodiment example, an antechamber 46. The antechamber 46 has a bypass opening 48 which can be manually sealed. In addition, the antechamber 46 has a cannula 50. Depending on the application, different cannulas can be mounted on the antechamber 46 or directly on the proximal portion 18. In this embodiment example, the means 34 having the antechamber 46 also comprise a prefilter 52 for filtering out connective tissue residues. The latter has a transmission limit of 1.5 mm.

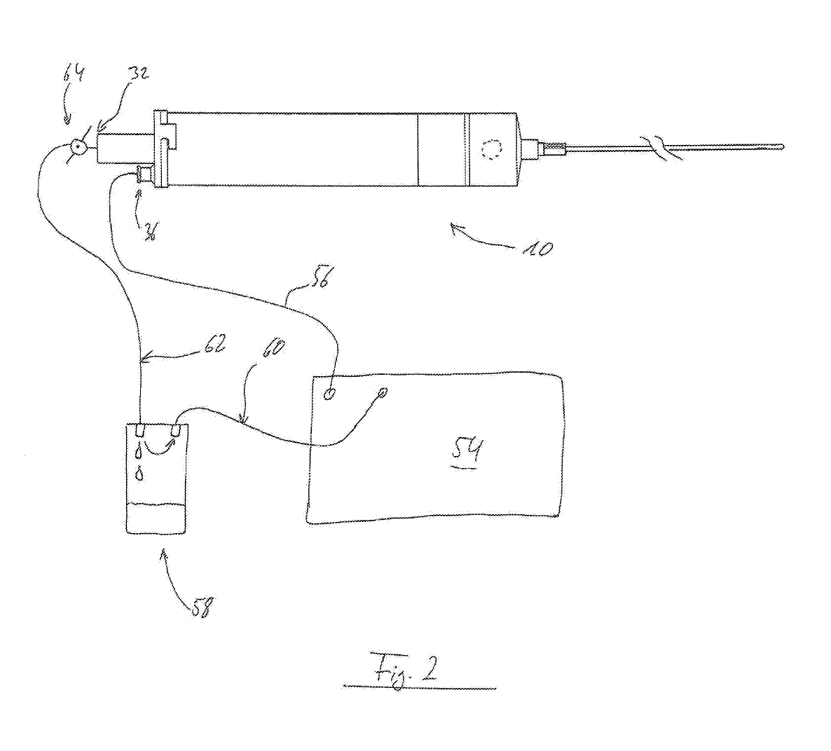

FIG. 2 shows a schematic representation of a system according to the invention, comprising at least one device according to the invention for transplanting body fat 10 and at least one media supply device 54. If reference numerals are used which are identical to those of FIG. 1, these describe the same features and apply at least additionally to FIG. 2 as well. FIG. 2 shows the device 10 and the media supply device 54. The media supply device 54 is connected by means of a first line 56 to the second media connection 36 of the device 10. The media supply device 54 is configured to supply the device 10 via the line 56 with ambient pressure or any positive pressure, but in particular between 0.5 and 30 bar. Furthermore, the system comprises means 58 for separating gaseous media flows and liquid material flows. The means 58 are switched between the device 10 and the media supply device 54 and are operatively connected therewith. The media supply device 54 is connected by means of a second line 60 and a third line 62 to the device 10 via the means 58. The line 62 is thereby connected to the first closable media connection 32 of the device 10. A switchable shut-off valve 64 is shown in principle here. The media supply device 54 is configured to supply the device 10 via the lines 60, 62 with ambient pressure or any negative pressure, but in particular between −900 mbar and −500 mbar.

### LIST OF REFERENCE NUMERALS

10 Device for transplanting body fat
12 Chamber in the form of a hollow cylinder
14 Plunger
16 Inner casing surface
18 Proximal portion
20 Distal portion
22 Sealing lips
24 Piston
26 Distal end
28 Fluidic connection
30 Filter
32 First closable media connection
34 Means for guiding fluids and/or fatty tissue
36 Second media connection
38 Additional fluidic connection
40 Outer casing surface
42 Outlet opening
44 Valve
46 Antechamber
48 Bypass opening
50 Cannula
52 Prefilter
54 Media supply device
56 Line
58 Means for separating gaseous media flows and liquid material flows
60 Line
62 Line
64 Switchable shut-off valve

1. A device for transplanting body fat, comprising a chamber which, at least in sections, is in the form of a hollow cylinder, said chamber being divided by a plunger which can be guided on an inner casing surface of the chamber into a proximal portion and a distal portion which is fluidically decoupled from said proximal portion, wherein a piston is connected to the plunger, said piston comprising a distal end which has a fluidic connection extending through the plunger to the proximal portion of the chamber, and the plunger is configured, at least in sections, as a filter in the region of the fluidic connection, and the distal end of the piston comprises a first closable media connection and, furthermore, means for guiding fluids and/or fatty tissue are or can be connected to the proximal portion of the chamber, wherein fluid and/or fatty tissue can be sucked into the proximal portion of the chamber and/or fluid can be sucked in through the plunger by supplying the first media connection in the open state with a negative pressure,
wherein
the distal portion of the chamber comprises a second media connection, so that fluid and/or fatty tissue can be pumped out by means of a displacement of the plunger through the proximal portion of the chamber by supplying the second media connection with a positive pressure while the first media connection is closed,
and/or that fluid and/or fatty tissue can be pumped out by means of the displacement of the plunger through the proximal portion of the chamber and, simultaneously, fluid can be discharged through the plunger by supplying the second media connection with a positive pressure while the first media connection is at least open.

2. The device according to claim 1, wherein the plunger has at least one additional fluidic connection between an outer casing surface of the plunger and an outlet opening at the distal end of the piston.

3. The device according to claim 1, wherein at least one antechamber having at least one bypass opening and/or at least one cannula or cannula having a bypass opening is/are or can be connected to the proximal portion of the chamber as means for guiding fluids and/or fatty tissue.

4. The device according to claim 3, wherein the antechamber comprises at least one prefilter for filtering out connective tissue residues.

5. The device according to claim 1, wherein the second media connection comprises a valve which is in a flow-blocking state when the piston is located in a distal dead-center position, and is in a flow-enabling state when the piston is not located in a distal dead-center position.

6. The device according to claim 1, wherein the piston can be latched in a distal dead-center position by being rotated about its longitudinal axis.

7. The device according to claim 1, wherein a transmission limit of at least one portion of the plunger which is configured as a filter is between 100 μm and 300 μm and/or a transmission limit of at least one prefilter is between 0.5 mm and 3 mm.

8. The device according to claim 1, wherein the second media connection can be acted upon with a positive pressure between 0.5 and 30 bar and/or the first media connection can be acted upon with a negative pressure between −900 mbar and −300 mbar.

9. A system, comprising at least one device for transplanting body fat according to claim 1 and at least one media supply device.

10. The system according to claim 9, further comprising means for separating gaseous media flows and liquid material flows, which are arranged in an operative connection between the device for transplanting body fat and the media supply device.
